(19) United States
(12) Patent Application Publication
(10) Pub. No.: US 2004/0010150 A1
(43) Pub. Date: Jan. 15, 2004

(54) Process for making 2-amino-5-cyanothiazole compounds

(76) Inventor: Matthew M. Zhao, Edison, PA (US)

Correspondence Address: MERCK AND CO INC, P O BOX 2000, RAHWAY, NJ, 070650907

(21) Appl. No.: 10/607,117

(22) Filed: Jun. 26, 2003

Related U.S. Application Data
(60) Provisional application No. 60/395,922, filed on Jul. 15, 2002.

(51) Int. Cl. C07D 277/18

(52) U.S. Cl. 548/190

(57) ABSTRACT

The present invention relates to methods of preparing 2-amino-5-cyanothiazoles, which are useful as intermediates in the preparation of compounds that are known to be useful in the treatment of cancer and other disease by inhibiting, modulating and/or regulating signal transduction of both receptor-type and non-receptor type tyrosine kinases.

### BACKGROUND OF THE INVENTION

The present invention relates to a process for making 2-amino-5-cyanothiazole compounds, which are useful intermediates for synthesizing compounds that inhibit, regulate and/or modulate tyrosine kinase signal transduction, and may be used to treat tyrosine kinase-dependent diseases and conditions, such as angiogenesis, cancer, tumor growth, atherosclerosis, age related macular degeneration, diabetic retinopathy, inflammatory diseases, and the like in mammals.

Tyrosine kinases are a class of enzymes that catalyze the transfer of the terminal phosphate of adenosine triphosphate to tyrosine residues in protein substrates. Tyrosine kinases play critical roles in signal transduction for a number of cell functions via substrate phosphorylation. Though the exact mechanisms of signal transduction is still unclear, tyrosine kinases have been shown to be important contributing factors in cell proliferation, carcinogenesis and cell differentiation.

Tyrosine kinases can be categorized as receptor type or non-receptor type. Receptor type tyrosine kinases have an extracellular, a transmembrane, and an intracellular portion, while non-receptor type tyrosine kinases are wholly intracellular.

The receptor-type tyrosine kinases are comprised of a large number of transmembrane receptors with diverse biological activity. In fact, about twenty different subfamilies of receptor-type tyrosine kinases have been identified. One tyrosine kinase subfamily, designated the HER subfamily, is comprised of EGFR, HER2, HER3, and HER4. Ligands of this subfamily of receptors include epithileal growth factor, TGF-α, amphiregulin, HB-EGF, betacellulin and heregulin. Another subfamily of these receptor-type tyrosine kinases is the insulin subfamily, which includes INS-R, IGF-IR, and IR-R. The PDGF subfamily includes the PDGF-α and β receptors, CSFIR, c-kit and FLK-II. Then there is the FLK family which is comprised of the kinase insert domain receptor (KDR), fetal liver kinase-1 (FLK-1), fetal liver kinase-4 (FLK-4) and the fms-like tyrosine kinase-1 (flt-1). The PDGF and FLK families are usually considered together due to the similarities of the two groups. For a detailed discussion of the receptor-type tyrosine kinases, see Plowman et al., DN&P 7(6):334-339, 1994, which is hereby incorporated by reference.

The non-receptor type of tyrosine kinases is also comprised of numerous subfamilies, including Src, Frk, Btk, Csk, Abl, Zap70, Fes/Fps, Fak, Jak, Ack, and LIMK. Each of these subfamilies is further sub-divided into varying receptors. For example, the Src subfamily is one of the largest and includes Src, Yes, Fyn, Lyn, Lck, Blk, Hck, Fgr, and Yrk. The Src subfamily of enzymes has been linked to oncogenesis. For a more detailed discussion of the non-receptor type of tyrosine kinases, see Bolen Oncogene, 8:2025-2031 (1993), which is hereby incorporated by reference.

Both receptor-type and non-receptor type tyrosine kinases are implicated in cellular signaling pathways leading to numerous pathogenic conditions, including cancer, psoriasis and hyperimmune responses.

Several receptor-type tyrosine kinases, and the growth factors that bind thereto, have been suggested to play a role in angiogenesis, although some may promote angiogenesis indirectly (Mustonen and Alitalo, J. Cell Biol. 129:895-898, 1995). One such receptor-type tyrosine kinase is fetal liver kinase 1 or FLK-1. The human analog of FLK-1 is the kinase insert domain-containing receptor KDR, which is also known as vascular endothelial cell growth factor receptor 2 or VEGFR-2, since it binds VEGF with high affinity. Finally, the murine version of this receptor has also been called NYK (Oelrichs et al., Oncogene 8(1):11-15, 1993). VEGF and KDR are a ligand-receptor pair that play an important role in the proliferation of vascular endothelial cells, and the formation and sprouting of blood vessels, termed vasculogenesis and angiogenesis, respectively.

Angiogenesis is characterized by excessive activity of vascular endothelial growth factor (VEGF). VEGF is actually comprised of a family of ligands (Klagsburn and D'Amore, Cytokine & Growth Factor Reviews 7:259-270, 1996). VEGF binds the high affinity membrane-spanning tyrosine kinase receptor KDR and the related fms-like tyrosine kinase-1, also known as Flt-1 or vascular endothelial cell growth factor receptor 1 (VEGFR-1). Cell culture and gene knockout experiments indicate that each receptor contributes to different aspects of angiogenesis. KDR mediates the mitogenic function of VEGF whereas Flt-1 appears to modulate non-mitogenic functions such as those associated with cellular adhesion. Inhibiting KDR thus modulates the level of mitogenic VEGF activity. In fact, tumor growth has been shown to be susceptible to the antiangiogenic effects of VEGF receptor antagonists. (Kim et al., Nature 362, pp. 841-844, 1993).

Solid tumors can therefore be treated by tyrosine kinase inhibitors since these tumors depend on angiogenesis for the formation of the blood vessels necessary to support their growth. These solid tumors include histiocytic lymphoma, cancers of the brain, genitourinary tract, lymphatic system, stomach, larynx and lung, including lung adenocarcinoma and small cell lung cancer. Additional examples include cancers in which overexpression or activation of Raf-activating oncogenes (e.g., K-ras, erb-B) is observed. Such cancers include pancreatic and breast carcinoma. Accordingly, inhibitors of these tyrosine kinases are useful for the prevention and treatment of proliferative diseases dependent on these enzymes.

The angiogenic activity of VEGF is not limited to tumors. VEGF accounts for most of the angiogenic activity produced in or near the retina in diabetic retinopathy. This vascular growth in the retina leads to visual degeneration culminating in blindness. Ocular VEGF MRNA and protein are elevated by conditions such as retinal vein occlusion in primates and decreased pO₂ levels in mice that lead to neovascularization. Intraocular injections of anti-VEGF monoclonal antibodies or VEGF receptor immunofusions inhibit ocular neovascularization in both primate and rodent models. Regardless of the cause of induction of VEGF in human diabetic retinopathy, inhibition of ocular VEGF is useful in treating the disease.

Expression of VEGF is also significantly increased in hypoxic regions of animal and human tumors adjacent to areas of necrosis. VEGF is also upregulated by the expression of the oncogenes ras, raf, src and mutant p53 (all of which are relevant to targeting cancer). Monoclonal anti-VEGF antibodies inhibit the growth of human tumors in nude mice. Although these same tumor cells continue to express VEGF in culture, the antibodies do not diminish their mitotic rate. Thus tumor-derived VEGF does not function as an autocrine mitogenic factor. Therefore, VEGF contributes to tumor growth in vivo by promoting angiogenesis through its paracrine vascular endothelial cell chemotactic and mitogenic activities. These monoclonal antibodies also inhibit the growth of typically less well vascularized human colon cancers in athymic mice and decrease the number of tumors arising from inoculated cells.

Viral expression of a VEGF-binding construct of Flk-1, Flt-1, the mouse KDR receptor homologue, truncated to eliminate the cytoplasmic tyrosine kinase domains but retaining a membrane anchor, virtually abolishes the growth of a transplantable glioblastoma in mice presumably by the dominant negative mechanism of heterodimer formation with membrane spanning endothelial cell VEGF receptors. Embryonic stem cells, which normally grow as solid tumors in nude mice, do not produce detectable tumors if both VEGF alleles are knocked out. Taken together, these data indicate the role of VEGF in the growth of solid tumors. Inhibition of KDR or Flt-1 is implicated in pathological angiogenesis, and these receptors are useful in the treatment of diseases in which angiogenesis is part of the overall pathology, e.g., inflammation, diabetic retinal vascularization, as well as various forms of cancer since tumor growth is known to be dependent on angiogenesis. (Weidner et al., N. Engl. J. Med., 324, pp. 1-8, 1991).

A number of compounds have been identified as inhibiting tyrosine kinase signal transduction, in particular as inhibitors of KDR. Several of these KDR inhibitors are characterized by an amino-cyanothiazole moiety, such as those illustrated in PCT Publications WO 01/17995 (published Mar. 15, 2001) and WO 02/45652 (published 6/13/2002).

Accordingly, a practical, efficient synthesis of amino-cyanothiazolyl intermediates is desirable and is an object of this invention.

### SUMMARY OF THE INVENTION

The present invention relates to methods of preparing compounds that are synthetic intermediates of pharmaceutical compounds that are capable of inhibiting, modulating and/or regulating signal transduction of both receptor-type and non-receptor type tyrosine kinases.

### DETAILED DESCRIPTION OF THE INVENTION

The instant invention is related to a process for preparing an unsubstituted or substituted 2-amino-5-cyanothiazole compounds or its pharmaceutically acceptable salt, such as illustrated by Formula I

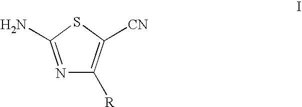

wherein

R is H, unsubstituted or substituted C₁-C₁₀ alkyl, unsubstituted or substituted aryl or unsubstituted or substituted heteroaryl;

which comprises the steps of:

a) halogenating and hydrolyzing a solution of an unsubstituted or substituted 3-alkoxy or 3-aryloxy acrylonitrile in a solvent to produce a mixture;

b) adding thiourea to the mixture and neutralizing to produce a product; and

c) isolating the amino-cyanothiazole of Formula I.

Another embodiment of the instant invention is the above described process which comprises:

a) brominating and hydrolyzing a solution of a substituted or unsubstituted 3-methoxyacrylonitrile of Formula II

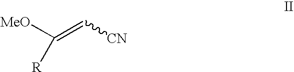

(wherein R is defined above) in acetonitrile to produce a mixture;

b) adding thiourea to the mixture and neutralizing produce a product; and

c) isolating the amino-cyanothiazole.

A further embodiment of the instant invention is the above described process which further comprises:

a) brominating and hydrolyzing a solution of a substituted or unsubstituted 3-methoxyacrylonitrile of Formula II

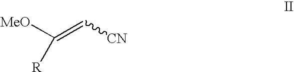

(wherein R is defined above) in acetonitrile to produce a mixture;

b) adding thiourea to the mixture, neutralizing and heating to a temperature of about 40° C. to about 70° C. to produce a product; and

c) isolating the amino-cyanothiazole.

A preferred embodiment of the instant invention is a process for preparing an amino-cyanothiazole of Formula Ia

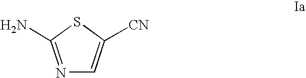

which comprises the steps of:

a) brominating and hydrolyzing a solution of 3-methoxyacrylonitrile in acetonitrile to produce a mixture;

b) adding thiourea to the mixture;

c) adding NaOAc to neutralize the mixture; and isolating the amino-cyanothiazole of Formula Ia.

These and other aspects of the invention will be apparent from the teachings contained herein.

“Tyrosine kinase-dependent diseases or conditions” refers to pathologic conditions that depend on the activity of one or more tyrosine kinases. Tyrosine kinases either directly or indirectly participate in the signal transduction pathways of a variety of cellular activities including proliferation, adhesion and migration, and differentiation. Diseases associated with tyrosine kinase activities include the proliferation of tumor cells, the pathologic neovascularization that supports solid tumor growth, ocular neovascularization (diabetic retinopathy, age-related macular degeneration, and the like) and inflammation (psoriasis, rheumatoid arthritis, and the like).

The compounds prepared by the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted.

When any substituent and/or variable occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” or “alkylene” are intended to include both branched and unbranched, cyclic and acyclic saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement and may be cyclic or acyclic. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, cyclopropyl, methyl-cyclopropyl, 2,2-dimethyl-cyclobutyl, 2-ethyl-cyclopentyl, cyclohexyl, and so on. In some instances, definitions may appear for the same variable reciting both alkyl and cycloalkyl when a different number of carbons is intended for the respective substituents. The use of both terms in one definition should not be interpreted to mean in another definition that “alkyl” does not encompass “cycloalkyl” when only “alkyl” is used.

“Alkoxy” represents an alkyl group of 1 to 10 carbon atom, unless otherwise specified, as defined above attached through an oxygen bridge.

If no number of carbon atoms is specified, the term “alkenyl” refers to a non-aromatic hydrocarbon radical, which may be branched or unbranched and cyclic or acyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl, cyclohexenyl, methylenylcyclohexenyl, and so on.

The term “alkynyl” refers to a hydrocarbon radical, which may be branched or unbranched and cyclic or acyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃) CH₂CH(CH₃)Ph, and so on.

As used herein, “aryl” is intended to mean phenyl and substituted phenyl, including moieties with a fused benzo group. Examples of such aryl elements include phenyl, naphthyl, tetrahydronaphthyl, indanyl, biphenyl, phenanthryl, anthryl or acenaphthyl. In cases where the aryl substituent is bicyclic, it is understood that attachment is via the phenyl ring. Unless otherwise indicated, “aryl” includes phenyls substituted with one or more substituents.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

As appreciated by those of skill in the art, “halo” or “halogen” as used herein is intended to include chloro, fluoro, bromo and iodo.

The term “heterocycle” or “heterocyclyl” as used herein is intended to mean a 5- to 10-membered aromatic or nonaromatic heterocycle containing from 1 to 4 heteroatoms selected from the group consisting of 0, N and S, and includes bicyclic groups. “Heterocyclyl” therefore includes the above mentioned heteroaryls, as well as dihydro and tetrathydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, aziridinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

The alkyl, alkenyl, alkynyl, cycloalkyl, aryl, heteroaryl and heterocyclyl substituents may be unsubstituted or unsubstituted, unless specifically defined otherwise. For example, a (C₁-C₆)alkyl may be substituted with one, two or three substituents selected from F, Cl, Br, CF₃, N₃, NO₂, NH₂, oxo, —OH, —O(C₁-C₆ alkyl), S(O)₀₋₂, (C₁-C₆ alkyl) S(O)₀₋₂—, (C₁-C₆ alkyl)S(O)₀₋₂(C₁-C₆ alkyl)—, C₃-C₁₀ cycloalkyl, C₂-C₆ alkenyl, C₂-C₆ alkynyl, —C(O)NH, (C₁-C₆ alkyl) C(O)NH—, H₂NC(NH)—, (C₁-C₆ alkyl)C(O)—, —O(C₁-C₆ alkyl)CF₃, (C₁-C₆ alkyl)OC(O)—, (C₁-C₆ alkyl)O(C₁-C₆ alkyl)—, (C₁-C₆ alkyl)C(O)₂(C₁-C₆ alkyl)-, (C₁-C₆ alkyl)OC(O)NH—, aryl, benzyl, heterocycle, aralkyl, heterocyclylalkyl, halo-aryl, halo-benzyl, halo-heterocycle, cyano-aryl, cyano-benzyl and cyano-heterocycle. In this case, if one substituent is oxo and the other is OH, the following are included in the definition: —(C═O)CH₂CH(OH)CH₃, —(C═O)OH, —CH₂(OH)CH₂CH(O), and so on.

Some of the abbreviations that may be used in the description of the chemistry and in the Examples include:

| | |
|---|---|
| ACN | Acetonitrile; |
| Ac₂O | Acetic anhydride; |
| AcOH | Acetic acid; |
| AIBN | 2,2′-Azobisisobutyronitrile; |
| BINAP | 2,2′-Bis(diphenylphosphino)-1,1′ binaphthyl; |
| Bn | Benzyl; |
| BOC/Boc | tert-Butoxycarbonyl; |
| BSA | Bovine Serum Albumin; |
| CAN | Ceric Ammonia Nitrate; |
| CBz | Carbobenzyloxy; |
| CI | Chemical Ionization; |
| DBA | dibenzanthracene; |
| DBAD | Di-tert-butyl azodicarboxylate; |
| DBU | 1,8-Diazabicyclol[5.4.0]undec-7-ene; |
| DCE | 1,2-Dichloroethane; |
| DEAD | diethylazodicarboxylate; |
| DEM | diethoxymethane; |
| DIAD | diisopropylazodicarboxylate; |
| DIEA | N,N-Diisopropylethylamine; |
| DMAC | N,N-dimethylacetamide; |
| DMAP | 4-Dimethylaminopyridine; |
| DME | 1,2-Dimethoxyethane; |
| DMF | N,N-Dimethylformamide; |
| DMPU | 1,3-Dimethyl-3,4,5,6-tetrahydro-2-(1H)-pyrimidinone; |
| DMSO | Methyl sulfoxide; |
| DPAD | dipiperidineazodicarbonyl; |
| DPPA | Diphenylphosphoryl azide; |
| DTT | Dithiothreitol; |
| EDC | 1-(3-Dimethylaminopropyl)-3-ethyl- |
| | carbodiimide-hydrochloride; |
| EDTA | Ethylenediaminetetraacetic acid; |
| ES | Electrospray; |
| ESI | Electrospray ionization; |
| Et₂O | Diethyl ether; |
| Et₃N | Triethylamine; |
| EtOAc | Ethyl acetate; |
| EtOH | Ethanol; |
| FAB | Fast atom bombardment; |
| HEPES | 4-(2-Hydroxyethyl)-1-piperazineethanesulfonic acid; |
| HOAc | Acetic acid; |
| HMTA | Hexamethylenetetramine; |
| HOBT | 1-Hydroxybenzotriazole hydrate; |
| HOOBT | 3-Hydroxy-1,2,2-benzotriazin-4(3H)-one; |
| HPLC | High-performance liquid chromatography; |
| HRMS | High Resolution Mass Spectroscopy; |
| KOtBu | Potassium tert-butoxide; |
| LAH | Lithium aluminum hydride; |
| LCMS | Liquid Chromatography Mass Spectroscopy; |
| MCPBA | m-Chloroperoxybenzoic acid; |
| Me | Methyl; |
| MEK | Methyl ethyl ketone; |
| MeOH | Methanol; |
| MIBK | Methyl isobutyl ketone; |
| Ms | Methanesulfonyl; |
| MS | Mass Spectroscopy; |
| MsCl | Methanesulfonyl chloride; |
| MsOH | methanesulfonic acid; |
| MTBE | tert-butyl methyl ether; |
| n-Bu | n-butyl; |
| n-Bu₃P | Tri-n-butylphosphine; |
| NaHMDS | Sodium bis(trimethylsilyl)amide; |
| NBS | N-Bromosuccinimide; |
| NMP | N-Methyl pyrrolidinone; |
| ODCB | Ortho Dichlorobenzene, or 1,2-dichlorobenzene; |
| Pd(PPh₃)₄ | Palladium tetrakis(triphenylphosphine); |
| Pd₂(dba)₂ | Tris(dibenzylideneacetone)dipalladium (0) |
| Ph | phenyl; |
| PMSF | α-Toluenesulfonyl fluoride; |
| Py or pyr | Pyridine; |
| PYBOP | Benzotriazol-1-yloxytripyrrolidinophosphonium |
| (or PyBOP) | hexafluorophosphate; |
| RPLC | Reverse Phase Liquid Chromatography; |
| rt (or RT) | Room Temperature; |
| t-Bu | tert-Butyl; |
| TBAF | Tetrabutylammonium fluoride; |
| TBSCl | tert-Butyldimethylsilyl chloride; |
| TFA | Trifluoroacetic acid; |
| THF | Tetrahydrofuran; |
| TIPS | Triisopropylsilyl; |
| TM EDA | N,N,N′,N′-Tetramethylethylenediamine; |
| TMS | Tetramethylsilane; |
| Tr | Trityl; and |
| TsOH | P-Toluenesulfonic acid. |

The instant invention is directed to the preparation of a compound of Formula I which comprises the steps of halogenating and hydrolyzing a solution of an unsubstituted or substituted 3-alkoxy or 3-aryloxy acrylonitrile in a solvent to produce a mixture. A thiourea is added to the mixture and is then neutralized to produce a product. The compound of Formula I is then isolated.

As used herein, “halogenating” may be done by the addition of a halogenating agent to a solution or mixture in order to attach a halo or halogen to a compound. Halogenating agents may include, but are not limited to Br₂, NBS, 1,3-dibromo-5,5-dimethylhydantoin, pyr.HBr₃, NCS, Cl₂, 1,3-dichloro-5,5-dimethylhydantoin, pyr.HCl₃, F₂, 1,3-difluro-5,5-dimethylhydantoin and the like, to a solution or mixture. Most preferably, the instant process comprises the step of brominating a solution by adding a “brominating agent”, such as Br₂, NBS, 1,3-dibromo-5,5-dimethylhydantoin, pyr.HBr₃, and the like.

In the instant invention, a substituted or unsubstituted 3-alkoxy or a substituted or unsubstituted 3-aryloxy-acrylonitrile may be utilized. The substituted acrylonitrile may be substituted with a substituent selected from H, C₁-C₁₀ alkyl, aryl and heteroaryl groups. For example the unsubstituted or substituted 3-methoxy-acrylonitrile may be represented as

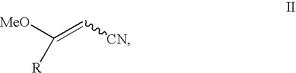

where R is H, unsubstituted or substituted C₁-C₁₀ alkyl, unsubstituted or substituted aryl or unsubstituted or substituted heteroaryl.

As used herein, the term “thiourea” may represent

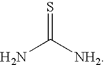

In the instant invention, the “neutralizing” step is done by adding NaOAc, NaH₂PO₄, Na₂HPO₄, NaHCO₃, K₂CO₃, KHCO₃, KOAc, KH₂PO₄, K₂HPO₄, and the like, to the mixture in order to adjust the pH of the mixture.

The use of the process of the instant invention to prepare intermediates that are useful making KDR inhibitors (such as those described in PCT Publications WO 01/17995 (published Mar. 15, 2001) and WO 02/45652 (published Jun. 13, 2002)) is illustrated in the following schemes, in addition to other standard manipulations that are known in the literature or exemplified in the experimental procedures. These schemes, therefore, are not limited by the compounds listed or by any particular substituents employed for illustrative purposes.

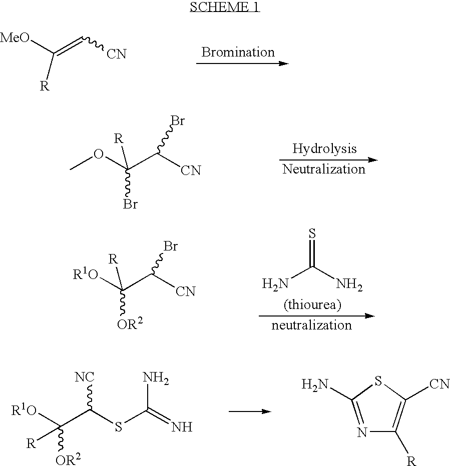

where R is as defined in Formula I and R¹ and R² independently represent H, or CH₃

### EXAMPLES

Examples provided are intended to assist in a further understanding of the invention. Particular materials employed, species and conditions are intended to be further illustrative of the invention and not limiting of the reasonable scope thereof.

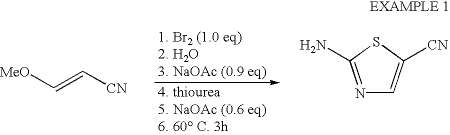

Bromine (2.88 Kg, 18.0 mole) is added to a solution of 3-methoxyacrylonitrile (1.50 Kg, 18.0 mole, mixture of cis-/trans-isomers) in acetonitrile (3.00 L) at 5-10° C. The mixture is aged for 20 minutes, then pre-cooled water (˜5° C., 12.0 L) is added and vigorous stirred for 1 hour.

NaOAc.3H₂O, (2.21 Kg, 16.2 mole, 0.90 equiv.) is added and stirred for 15 minutes and then thiourea (1.51 Kg, 19.80 mole, 1.10 equiv.) is added (endothermic dissolution followed by ˜10-15° C. exotherm in ˜0.5 h). The mixture is aged at 15° C. for 1.5 hour, then more NaOAc.3H₂O (1.47 Kg, 0.60 equiv.) is added. It is slowly heated to 60° C. in 1 hour and aged for 3 hours at 60° C. then cooled to 10° C.

NaOH (10 N, 1.13 L, 0.625 equiv.) is added to adjust the pH to 3.8-4.0. After aging for 1 hour, the product is filtered and washed with water (11.5 L). Drying give 1.86 Kg of the crude aminothiazole as a brown solid, (97A %), 80.7% yield corrected for 97.6w % purity.

The crude product is dissolved into acetone (35 L) at 50° C. and treated with Darco KB-B (380 g) for 2 hours. It is filtered through a Solka-Floc pad and then rinsed with acetone (5 L). The filtrate is concentrated in vacuo to ˜7 L (˜5 L residue acetone).

Heptane (10 L) is added in 0.5 hour and the slurry is aged for 1 hour. The product is filtered and the filter cake is washed with 2/1 heptane/acetone (6 L). Drying at rt affords 1.72 Kg of the aminothiazole as a pinkish solid, 75% yield corrected for 98.5w % purity.

HPLC conditions: Ace-C8 4.6×250 mm column; linear gradient: 5-80% MeCN in 12 5 minutes, 0.1% H₃PO₄ in the aqueous mobile phase; Flow rate: 1.50 ml/min; UV detection at 220 nm.

What is claimed is:

1. A process for preparing an unsubstituted or substituted 2-amino-5-cyanothiazole compounds of Formula I 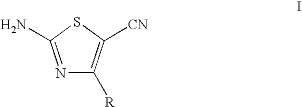
or a pharmaceutically acceptable salt,
wherein
R is H, unsubstituted or substituted C₁-C₁₀ alkyl, unsubstituted or substituted aryl or unsubstituted or substituted heteroaryl;
which comprises the steps of:
a) halogenating and hydrolyzing a solution of an unsubstituted or substituted 3-alkoxy or 3-aryloxy acrylonitrile in a solvent to produce a mixture;
b) adding thiourea to the mixture and neutralizing to produce a product; and
c) isolating the amino-cyanothiazole of Formula I.

2. The process according to claim 1 which comprises:
a) brominating and hydrolyzing a solution of a substituted or unsubstituted 3-methoxyacrylonitrile of Formula II 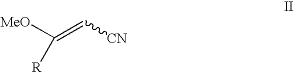
(where R is defined as in claim 1) in acetonitrile to produce a mixture;
b) adding thiourea to the mixture and neutralizing produce a product; and
c) isolating the amino-cyanothiazole.

3. The process according to claim 2 which comprises:
a) brominating and hydrolyzing a solution of a substituted or unsubstituted 3-methoxyacrylonitrile of Formula II 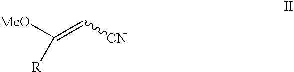
(where R is defined as in claim 1) in acetonitrile to produce a mixture;
b) adding thiourea to the mixture, neutralizing and heating to a temperature of about 40° C. to about 70° C. to produce a product; and
c) isolating the amino-cyanothiazole.

4. A process for preparing an amino-cyanothiazole of Formula Ia 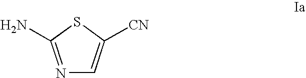
which comprises the steps of:
a) brominating and hydrolyzing a solution of 3-methoxyacrylonitrile in acetonitrile to produce a mixture;
b) adding thiourea to the mixture;
c) adding NaOAc to neutralize the mixture; and isolating the amino-cyanothiazole of Formula Ia.
